(19) United States
(12) Patent Application Publication
(10) Pub. No.: US 2015/0162533 A1
(43) Pub. Date: Jun. 11, 2015

(54) MATERIAL FOR ORGANIC ELECTROLUMINESCENCE DEVICE AND ORGANIC ELECTROLUMINESCENCE DEVICE HAVING THE SAME

(71) Applicant: SAMSUNG DISPLAY CO., LTD., Yongin-City (KR)

(72) Inventors: Hiroaki ITOI, Yokohama (JP); Yasuo MIYATA, Yokohama (JP)

(21) Appl. No.: 14/563,308

(22) Filed: Dec. 8, 2014

(30) Foreign Application Priority Data
Dec. 9, 2013 (JP) 2013-254541

(51) Int. Cl. H01L 51/00; C07C 211/61

(52) U.S. Cl. CPC H01L 51/006; C07C 211/61; C07C 2103/42; H01L 51/0054; H01L 51/0058; H01L 51/5221

(57) ABSTRACT

A material for an organic electroluminescence (EL) device, the material including a compound represented by the following Formula (1):

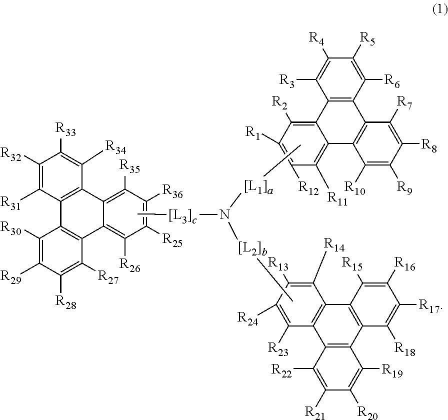

### CROSS-REFERENCE TO RELATED APPLICATION

Japanese Patent Application No. 2013-254541, filed on Dec. 9, 2013, in the Japanese Patent Office, and entitled: “Material for Organic Electroluminescence Device and Organic Electroluminescence Device Having the Same,” is incorporated by reference herein in its entirety.

### BACKGROUND

1. Field

Embodiments relate to a material for an organic electroluminescence device and an organic electroluminescence device having the same.

2. Description of the Related Art

Organic electroluminescence (EL) displays are one type of image displays that have been actively developed. Unlike a liquid crystal display and the like, the organic EL display is so-called a self-luminescent display which recombines holes and electrons injected from an anode and a cathode in an emission layer to thus emit light from a light-emitting material including an organic compound of the emission layer, thereby performing display.

### SUMMARY

Embodiments are directed to a material for an organic electroluminescence (EL) device, the material including a compound represented by the following Formula (1):

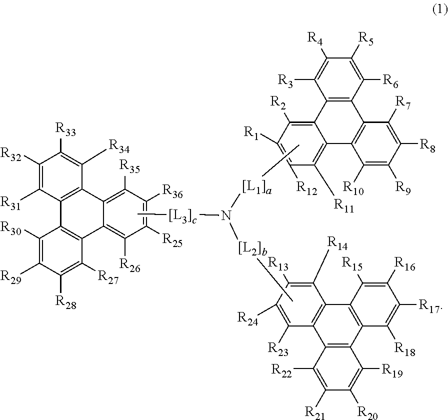

In Formula (1),

R₁ to R₃₆ may each independently be an aryl group having 6 to 30 ring carbon atoms, a heteroaryl group having 1 to 30 ring carbon atoms, an alkyl group having 1 to 15 carbon atoms, a hydrogen atom, a deuterium atom, or a bonding site at which N or a respective one of L₁ to L₃ is bound to a triphenylene ring carbon, and

L₁ to L₃ may each independently be a divalent connecting group, where L₁ is combined with one of R₁ to R₁₂, L₂ is combined with one of R₁₃ to R₂₄, and L₃ is combined with one of R₂₅ to R₃₆, and a, b, and c may each independently be an integer from 0 to 3, and may satisfy the equation 1≦a+b+c.

L₁ to L₃ of Formula (1) may each be a phenylene group and the compound may be represented by the following Formula (2):

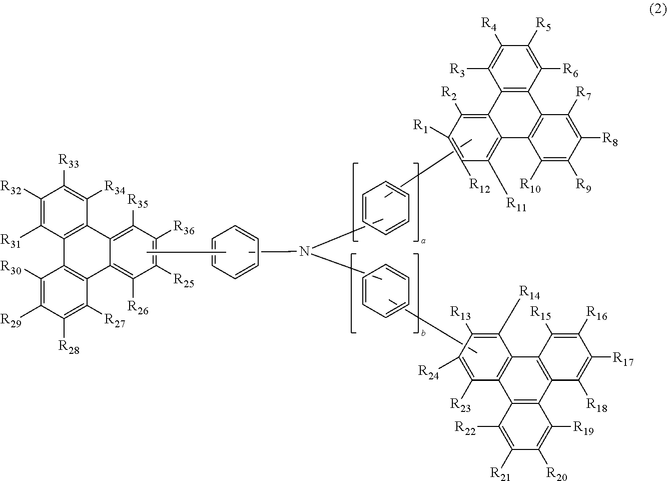

In Formula (1), a, b, and c may satisfy the equation 1≧a+b+c≧2.

Embodiments are also directed to an organic electroluminescence (EL) device including the material according to an embodiment in a layer between an emission layer and an anode.

Embodiments are also directed to an organic electroluminescence (EL) device including the material according to an embodiment in an emission layer.

### BRIEF DESCRIPTION OF THE DRAWING

Features will become apparent to those of skill in the art by describing in detail example embodiments with reference to the attached drawing, in which:

FIG. 1 illustrates a schematic diagram of an organic EL device according to an example embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

A material for an organic EL device according to an example embodiment includes a compound that is an amine derivative in which triphenylene is introduced near an amine part, as represented in the following Formula (1).

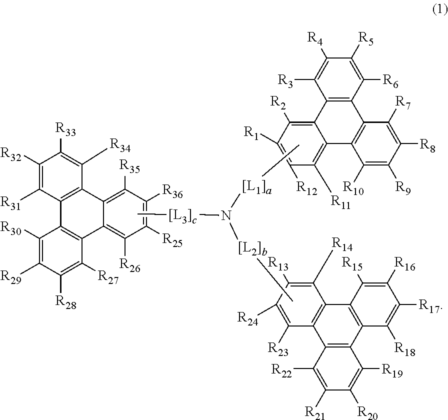

According to the present example embodiment, in Formula 1, R₁ to R₃₆ are independently an aryl group having 6 to 30 ring carbon atoms, a heteroaryl group having 1 to 30 ring carbon atoms, an alkyl group having 1 to 15 carbon atoms, a hydrogen atom, a deuterium atom, or a bonding site to which N or a respective one of L₁ to L₃ is bound to a triphenylene ring carbon. Also, each of L₁ to L₃ is a divalent connecting group, where L₁ is combined with one of R₁ to R₁₂, L₂ is combined with one of R₁₃ to R₂₄ and L₃ is combined with one of R₂₅ to R₃₆, and each of a, b, and c is an integer from 0 to 3, and satisfy the equation 1≧a+b+c. For example, when a is 0, N may be bound to any of R₁ to R₁₂, or any of R₁ to R₁₂ may be a bonding site where a single bond joins N to a triphenylene ring carbon. As another example, when a is 1, L₁ may be bound to any of R₁ to R₁₂, or any of R₁ to R₁₂ may be a bonding site where a single bond joins L₁ to a triphenylene ring carbon.

The molecular weight of the compound represented by Formula (1) may be, e.g., from about 600 to about 1,000.

The divalent connecting groups L₁ to L₃ may independently be, e.g., an arylene group or a heteroarylene group. In an embodiment, the divalent connecting groups L₁ to L₃ may be a phenylene group, a naphthalene group, a thienylene group, etc. For example, L₁ to L₃ may be the phenylene group. The divalent connecting groups of L₁ to L₃ and a, b, and c may be selected in an appropriate range to decrease the symmetry of the whole molecule of the amine derivative represented by Formula (1) so as to restrain the crystallization of the amine derivative represented by Formula (1) and to maintain good layer properties.

A material for an organic EL device according to an example embodiment includes a compound having three triphenylene groups having strong electron tolerance near an amine part having hole transport properties. The material may provide improved hole transport properties and electron tolerance. The material may be used as a hole transport layer, which may help provide high efficiency and long life when applied in an organic EL device. According to an example embodiment, at least one divalent connecting group is present between the amine part and the triphenylene, which may help ease a layer formation process, and which may help expand a conjugated system of π electrons of the whole molecule. Thus, the stability of the molecule may be increased and the life of a device may be improved.

In the material for an organic EL device according to the present example embodiment, each of L₁ to L₃ in Formula (1) may be a phenylene group. The phenylene group may be substituted or unsubstituted. The material for an organic EL device according to the present example embodiment includes an amine derivative compound having triphenylene near an amine part. The compound may be represented by the following Formula (2), in which a divalent connecting group between the amine and the triphenylene is a phenylene group.

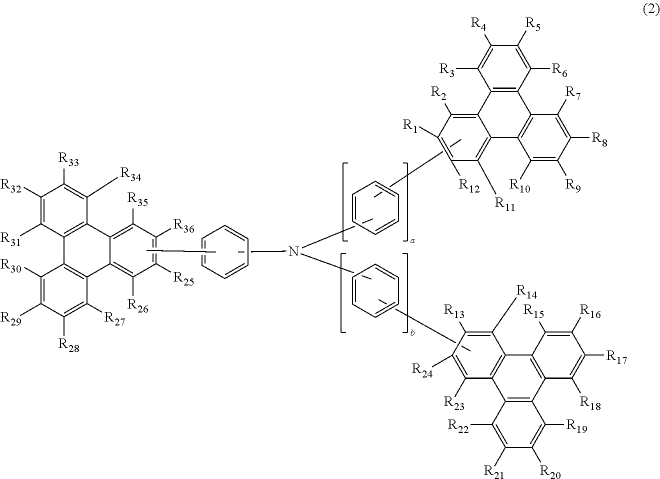

According to the present example embodiment, in Formula (2), R₁ to R₃₆ are independently an aryl group having 6 to 30 ring carbon atoms, a heteroaryl group having 1 to 30 ring carbon atoms, an alkyl group having 1 to 15 carbon atoms, a hydrogen atom, a deuterium atom, or a bonding site for N or a respective one of the triphenylene groups,

each of a, b, and c is an integer from 0 to 3, and

the equation 1≧a+b+c is satisfied.

The molecular weight of the compound represented by Formula (2) according to the present example embodiment may be from about 600 to about 1,000. In Formula (2), the phenylene group corresponding to L₁ of Formula (1) is combined with one of R₁ to R₁₂, the phenylene group corresponding to L₂ of Formula (1) is combined with one of R₁₃ to R₂₄, and the phenylene group corresponding to L₃ of Formula (1) is combined with one of R₂₅ to R₃₆. Values for a, b, and c may be selected from an appropriate range to restrain the crystallization of the amine derivative represented by Formula (2), to maintain good layer properties, and to decrease the symmetry of the whole molecule of the amine derivative represented by Formula (2).

The material for an organic EL device according to the present example embodiment includes a compound having three triphenylene groups with strong electron tolerance near an amine part with hole transport properties, which may help provide improved hole transport properties and electron tolerance. The material may be used to form a hole transport layer, which may help provide high efficiency and long life when used in an organic EL device. In addition, in the material for an organic EL device according to the present example embodiment, the compound has at least one divalent connecting group between the amine part and the triphenylene, e.g., a divalent phenyl group, in an amine derivative obtained by introducing the triphenylene in the amine part, which may help improve layer forming properties. With at least one phenylene group present between the amine part and the triphenylene, the conjugation system of π electrons of a whole molecule may be expanded, and the stability and the life of the device may be increased.

In the compounds represented by Formulae (1) and (2), a, b, and c in Formulae (1) and (2) may satisfy the equation 1≧a+b+c≦2 such that one or two divalent connecting groups, such as the phenylene group, are present between the amine and the triphenylene. Such a compound may be asymmetric, which may restrain crystallization of the material for an organic EL device during forming a layer, and which may increase amorphous properties. Thus, a hole transport layer having long life in an organic EL device may be provided.

The material for an organic EL device according to the present example embodiment may include, for example, one or more of the following compounds in accordance with Formula (1).

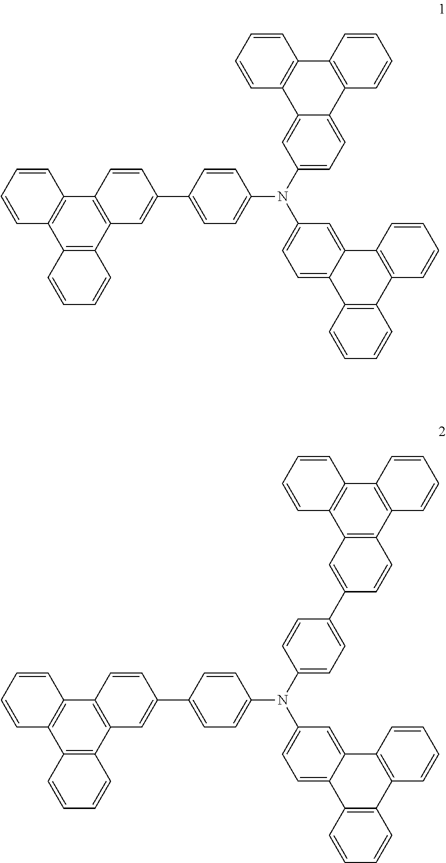 [SMILES: c1ccc2c(c1)c1ccccc1c1cc(-c3ccc(N(c4ccc5c6ccccc6c6ccccc6c5c4)c4ccc5c6ccccc6c6ccccc6c5c4)cc3)ccc21 ; c1ccc2c(c1)c1ccccc1c1cc(-c3ccc(N(c4ccc(-c5ccc6c7ccccc7c7ccccc7c6c5)cc4)c4ccc5c6ccccc6c6ccccc6c5c4)cc3)ccc21]

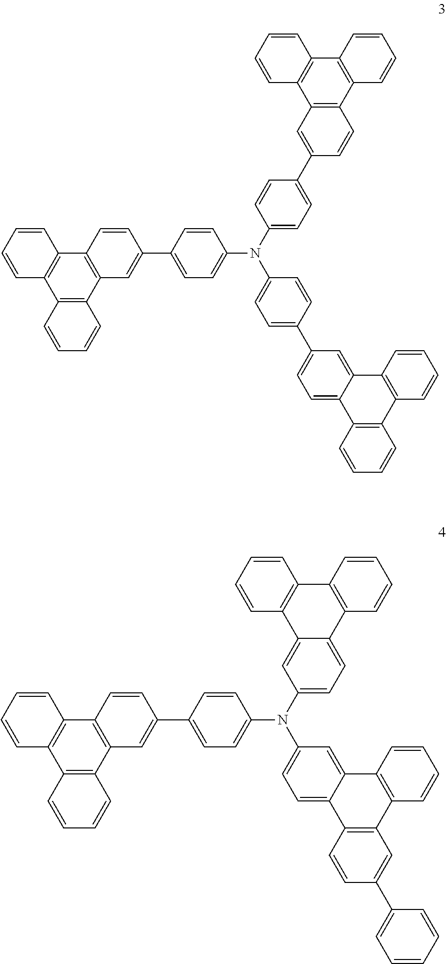 [SMILES: c1ccc2c(c1)c1ccccc1c1cc(-c3ccc(N(c4ccc(-c5ccc6c7ccccc7c7ccccc7c6c5)cc4)c4ccc(-c5ccc6c7ccccc7c7ccccc7c6c5)cc4)cc3)ccc21 ; c1ccc(-c2ccc3c4ccc(N(c5ccc(-c6ccc7c8ccccc8c8ccccc8c7c6)cc5)c5ccc6c7ccccc7c7ccccc7c6c5)cc4c4ccccc4c3c2)cc1]

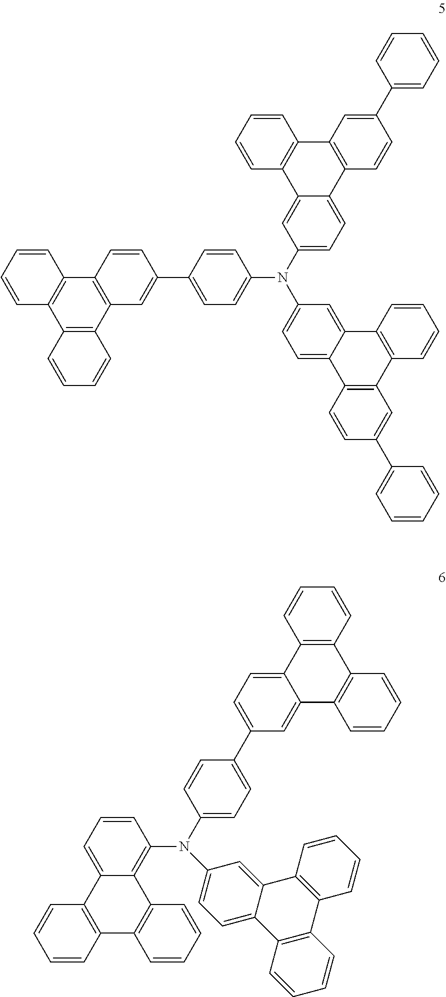 [SMILES: c1ccc(-c2ccc3c4ccc(N(c5ccc(-c6ccc7c8ccccc8c8ccccc8c7c6)cc5)c5ccc6c7ccc(-c8ccccc8)cc7c7ccccc7c6c5)cc4c4ccccc4c3c2)cc1 ; c1ccc2c(c1)c1ccccc1c1cc(-c3ccc(N(c4ccc5c6ccccc6c6ccccc6c5c4)c4cccc5c6ccccc6c6ccccc6c45)cc3)ccc21]

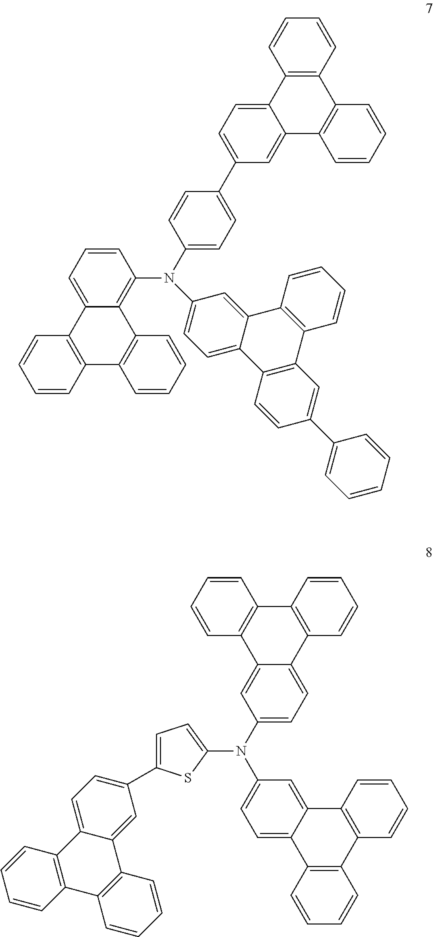 [SMILES: c1ccc(-c2ccc3c4ccc(N(c5ccc(-c6ccc7c8ccccc8c8ccccc8c7c6)cc5)c5cccc6c7ccccc7c7ccccc7c56)cc4c4ccccc4c3c2)cc1 ; c1ccc2c(c1)c1ccccc1c1cc(-c3ccc(N(c4ccc5c6ccccc6c6ccccc6c5c4)c4ccc5c6ccccc6c6ccccc6c5c4)s3)ccc21]

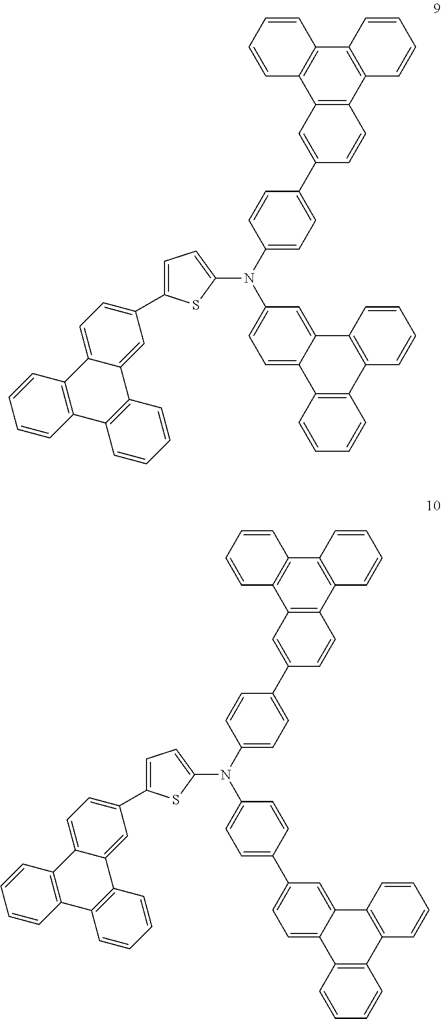 [SMILES: c1ccc2c(c1)c1ccccc1c1cc(-c3ccc(N(c4ccc5c6ccccc6c6ccccc6c5c4)c4ccc(-c5ccc6c7ccccc7c7ccccc7c6c5)s4)cc3)ccc21 ; c1ccc2c(c1)c1ccccc1c1cc(-c3ccc(N(c4ccc(-c5ccc6c7ccccc7c7ccccc7c6c5)cc4)c4ccc(-c5ccc6c7ccccc7c7ccccc7c6c5)s4)cc3)ccc21]

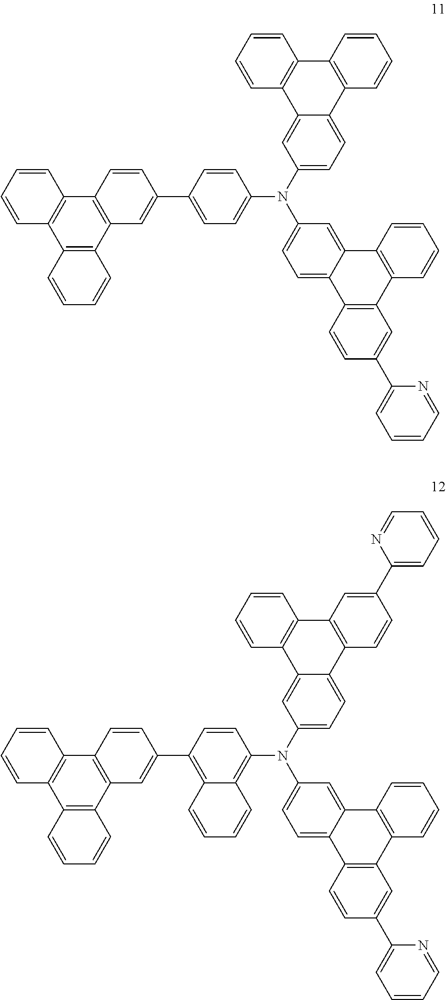 [SMILES: c1ccc(-c2ccc3c4ccc(N(c5ccc(-c6ccc7c8ccccc8c8ccccc8c7c6)cc5)c5ccc6c7ccccc7c7ccccc7c6c5)cc4c4ccccc4c3c2)nc1 ; c1ccc(-c2ccc3c4ccc(N(c5ccc6c7ccc(-c8ccccn8)cc7c7ccccc7c6c5)c5ccc(-c6ccc7c8ccccc8c8ccccc8c7c6)c6ccccc56)cc4c4ccccc4c3c2)nc1]

The material for an organic EL device according to an example embodiment may be used in a layer, e.g., among a plurality of stacked layers, disposed between an emission layer and an anode. The material for an organic EL device according to the present example embodiment may be also used in an emission layer of an organic EL device. Thus, the stability of a layer including the material for an organic EL device may be improved and the electron tolerance may be improved at the same time, which may help realize high efficiency and long life of an organic EL device. In addition, the material for an organic EL device according to the present example embodiment may be used in an emission layer or a layer of stacked layers disposed between the emission layer and an anode of an organic EL device in a blue emission region.

(Organic EL Device)

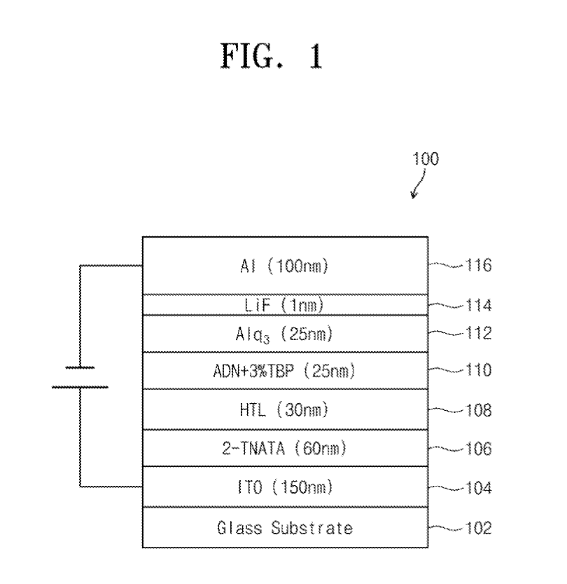

An organic EL device using the material for an organic EL device according to an example embodiment will be described in connection with FIG. 1, which schematically illustrates an organic EL device 100 according to an example embodiment.

Referring to FIG. 1, the organic EL device 100 according to the present example embodiment may include, for example, a substrate 102, an anode 104, a hole injection layer 106, a hole transport layer 108, an emission layer 110, an electron transport layer 112, an electron injection layer 114 and a cathode 116. The anode 104, the hole injection layer 106, the hole transport layer 108, the emission layer 110, the electron transport layer 112, the electron injection layer 114, and the cathode 116 may be stacked sequentially on the substrate 102. In an example embodiment, the material for an organic EL device according to an embodiment may be used in a layer of stacked layers disposed between the emission layer and the anode. In another example embodiment, the material for an organic EL device according to an embodiment may be used in the emission layer.

An example embodiment using the material for an organic EL device according to an example embodiment in the hole transport layer 108 will now be described. The substrate 102 may be a transparent glass substrate, a semiconductor substrate formed by using silicon, etc., or a flexible substrate of a resin, etc. The anode 104 is disposed on the substrate 102 and may be formed by using indium tin oxide (ITO), indium zinc oxide (IZO), etc. The hole injection layer 106 is disposed on the anode 104 and may include, for example, 4,4′,4″-tris(N-1-naphthyl-N-phenylamino)triphenylamine (1-TNATA), 4,4′,4″-tris(N-(2-naphthyl)-N-phenylamino)triphenylamine (2-TNATA), N,N,N′,N′-tetrakis(3-methylphenyl)-3,3′-dimethylbenzidine (HMTPD), etc. The hole transport layer 108 is disposed on the hole injection layer 106 and is formed using the material for an organic EL device according to an example embodiment. The emission layer 110 is disposed on the hole transport layer 108 and may be formed using, for example, a host material including 9,10-di(2-naphthyl)anthracene (ADN), etc. doped with tetra-t-butylperylene (TBP). The electron transport layer 112 is disposed on the emission layer 110 and may be formed using, for example, a material including tris(8-hydroxyquinolinato)aluminum (Alq₃). The electron injection layer 114 is disposed on the electron transport layer 112 and may be formed using, for example, a material including lithium fluoride (LiF). The cathode 116 is disposed on the electron injection layer 114 and may be formed using a metal such as Al or a transparent material such as indium tin oxide (ITO), indium zinc oxide (IZO), etc. The above-described thin layers may be formed using appropriate layer forming method such as vacuum deposition, sputtering, various coatings, etc.

In the organic EL device 100 according to the present example embodiment, a hole transport layer having high efficiency and long life may be formed by using the material for an organic EL device according to an embodiment. In addition, the material for an organic EL device according to an embodiment may be applied in an organic EL apparatus of an active matrix type using thin film transistors (TFT).

The organic EL device 100 according to the present embodiment includes the material for an organic EL device according to an embodiment in an emission layer or a layer of stacked layers disposed between the emission layer and an anode, which may help provide high efficiency and long life of the organic EL device.

(Synthetic Method)

A compound according to Formula (1) may be synthesized, for example, as follows.

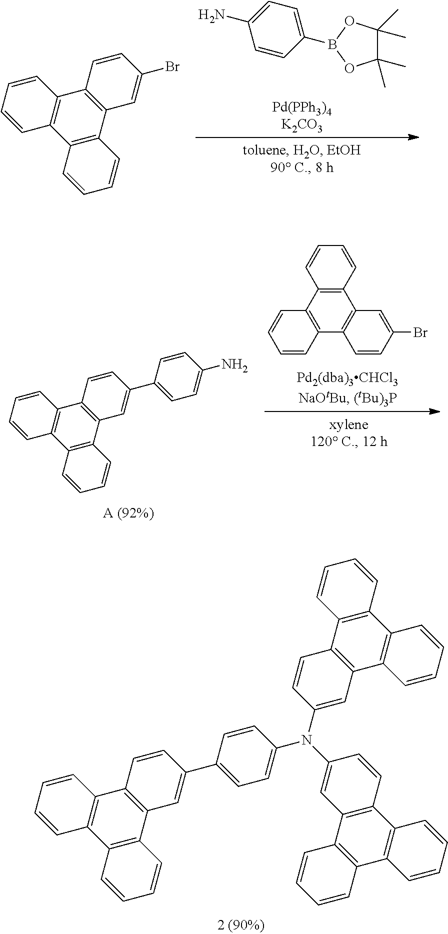 [SMILES: Brc1ccc2c3ccccc3c3ccccc3c2c1 ; CC1(C)OB(c2ccc(N)cc2)OC1(C)C ; Nc1ccc(-c2ccc3c4ccccc4c4ccccc4c3c2)cc1 ; Brc1ccc2c3ccccc3c3ccccc3c2c1 ; c1ccc2c(c1)c1ccccc1c1cc(-c3ccc(N(c4ccc5c6ccccc6c6ccccc6c5c4)c4ccc5c6ccccc6c6ccccc6c5c4)cc3)ccc21]

(Synthesis of Compound A)

5.00 g of 2-bromophenylene, 4.01 g of 4-(4,4,5,5-tetramethyl-1,3,2-dioxaborane-2-yl)aniline, 1.26 g of tetrakis(triphenylphosphine)palladium (Pd(PPh₃)₄), 131 mL of 2 M sodium carbonate (Na₂CO₃) aqueous solution, and 65 mL of ethanol were added in a 500 mL, four-necked flask under an argon atmosphere, followed by stirring in 325 mL of a toluene solvent at 90 degrees for 5 hours. After cooling in the air, an organic layer was separated, and solvents were distilled off. Then, recrystallization was performed using toluene to produce 4.68 g of Compound A as white solid (yield 92%).

(Synthesis of Compound 2)

1.50 g of Compound A, 2.74 g of 2-bromotriphenylene, 0.340 g of tris(dibenzilideneacetone)dipalladium(0) (Pd₂(dba)₃)-chloroform adduct, 0.150 g of tri-tert-butyl phosphine ((t-Bu)₃P) and 1.35 g of sodium tert-butoxide were added in a 500 mL, three-necked flask under an argon atmosphere, followed by stirring in 75 mL of a xylene solvent at 120 degrees for 10 hours. After cooling in the air, water was added in the flask, and an organic layer was separated. Activated charcoal was added in the organic layer, and filtering was performed in warm conditions. The solvents were distilled off, and the residue thus obtained was recrystallized using a THF/hexane mixture solvent to produce 3.27 g of Compound 2 as pale yellow solid (yield 90%).

(Identification method of compounds)

The identification of Compound A was conducted by measuring FAB-MS. The identification of Compound 2 was conducted by measuring ¹H-NMR and FAB-MS. CDCl₃ was used as a solvent for measuring ¹H-NMR.

(Identification of Compound A)

The molecular weight of Compound A measured by FAB-MS was 320.

(Identification of Compound 2)

Chemical shift values of Compound 2 measured by ¹H-NMR were 8.88 (d, 1H), 8.72-8.78 (m, 1H), 8.52-8.72 (m, 14H), 8.35 (d, 2H), 7.91 (d, 1H), 7.78 (d, 2H), 7.43-7.67 (m, 16H). In addition, the molecular weight of Compound 2 measured by FAB-MS was 772.

According to the above-described synthetic method, Compound 2 was prepared for a material for an organic EL device according to an embodiment. In addition, the following Comparative Compound 1, Comparative Compound 2, and Comparative Compound 3 were prepared for comparison.

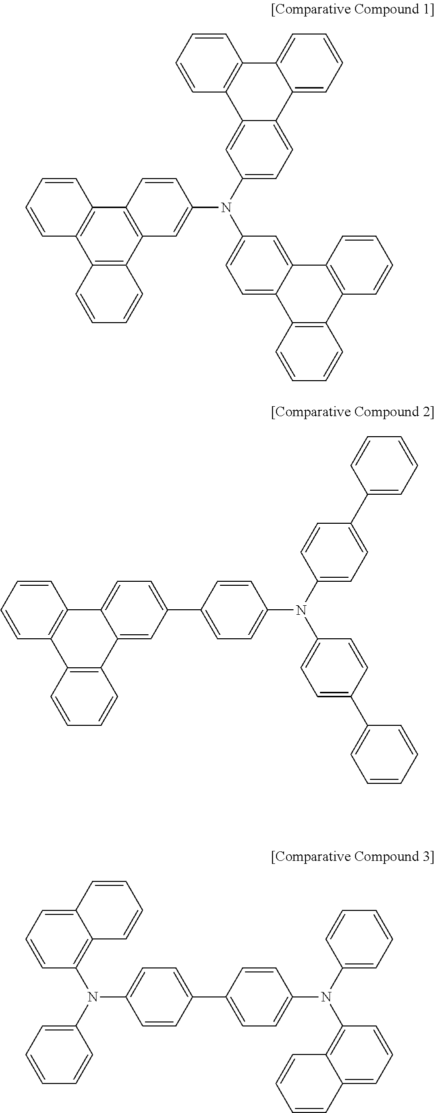 [SMILES: c1ccc2c(c1)c1ccccc1c1cc(N(c3ccc4c5ccccc5c5ccccc5c4c3)c3ccc4c5ccccc5c5ccccc5c4c3)ccc21 ; c1ccc(-c2ccc(N(c3ccc(-c4ccccc4)cc3)c3ccc(-c4ccc5c6ccccc6c6ccccc6c5c4)cc3)cc2)cc1 ; c1ccc(N(c2ccc(-c3ccc(N(c4ccccc4)c4cccc5ccccc45)cc3)cc2)c2cccc3ccccc23)cc1]

Organic EL devices were manufactured using Compound 2, Comparative Compound 1, Comparative Compound 2, and Comparative Compound 3 as hole transport materials for a hole transport layer. In these devices, the substrate 102 was formed using a transparent glass substrate, the anode 104 was formed using ITO to a thickness of about 150 nm, the hole injection layer 106 was formed using 2-TNATA to a thickness of about 60 nm, the hole transport layer 108 was formed to a thickness of about 30 nm, the emission layer 110 was formed using ADN doped with 3% TBP to a thickness of about 25 nm, the electron transport layer 112 was formed using Alq₃ to a thickness of about 25 nm, the electron injection layer 114 was formed using LiF to a thickness of about 1 nm, and the cathode 116 was formed using Al to a thickness of about 100 nm.

With respect to the organic EL devices thus manufactured, the voltage, the current efficiency, and the half life were evaluated. In this case, the current efficiency corresponds to values at the current density of 10 mA/cm², and the half life means luminance half life from an initial luminance of 1,000 cd/m². The evaluation results are illustrated in Table 1

TABLE 1

| | Voltage (V) | Current efficiency (cd/A) | Half life (hr) |
|---|---|---|---|
| Compound 2 | 4.7 | 7.3 | 3,300 |
| Comparative | 4.7 | 6.9 | 2,800 |
| Compound 1 | | | |
| Comparative | 6.5 | 6.2 | 1,500 |
| Compound 2 | | | |
| Comparative | 8.1 | 5.3 | 1,200 |
| Compound 3 | | | |

From the results in Table 1, it is seen that the organic EL device including Comparative Compound 1 had a lower driving voltage and had higher current efficiency and longer life when compared to the organic EL devices including Comparative Compound 2 and Comparative Compound 3.

In addition, the organic EL device including Compound 2 as the material for an organic EL device in accordance with an embodiment was driven at a lower voltage when compared to the organic EL devices including Comparative Compounds 2 and 3. With respect to the current efficiency, the organic EL device including Compound 2 as the material for an organic EL device in accordance with an embodiment had higher current efficiency when compared to the organic EL devices including Comparative Compounds 1, 2, and 3.

Without being bound by theory, it is believed that Comparative Compound 1, having three triphenylene groups having strong electron tolerance, provided higher electron tolerance when compared to Comparative Compound 2 including one triphenylene group and Comparative Compound 3 including no triphenylene group.

Without being bound by theory, it is believed that Compound 2, having three triphenylene groups having strong electron tolerance near an amine part, provided improved hole transport properties and electron tolerance. In Compound 2, at least one divalent connecting group is present between an amine part and triphenylene. Without being bound by theory, it is believed that the conjugation system of π electrons in the whole molecule of Compound 2 is expanded, helping to improve the stability and the life of the organic EL device formed using the material including Compound 2 according to an embodiment.

In the material for an organic EL device according to the present example embodiment, triphenylene is introduced near an amine part in a compound, which may help improve hole transport properties and electron tolerance. Further, a hole transport layer having high efficiency and long life may be formed when applied in an organic EL device.

By way of summation and review, an organic electroluminescence device (organic EL device) may include, e.g., an anode, a hole transport layer disposed on the anode, an emission layer disposed on the hole transport layer, an electron transport layer disposed on the emission layer, and a cathode disposed on the electron transport layer. Holes injected from the anode are injected into the emission layer via the hole transport layer. Meanwhile, electrons are injected from the cathode, and then injected into the emission layer via the electron transport layer. The holes and the electrons injected into the emission layer are recombined to generate excitons within the emission layer. The organic EL device emits light by using light generated during the transition of the excitons to a ground state.

In the application of the organic EL device in a display apparatus, high efficiency and long life of the organic EL device are desirable. For the realization of high efficiency and long life of the organic EL device, the normalization, the stabilization, and the durability of a hole transport layer have been examined.

As described above, embodiments relate to a material for an organic electroluminescence device having high efficiency and long life, and an organic electroluminescence device using the same. Embodiments may provide an organic EL device having long life and the high efficiency. A material used in the organic EL device may include a compound having triphenylene near an amine part.

In the organic EL device according to an embodiment, hole transport properties and electron tolerance may be improved, and long life and high efficiency may be realized when using a material for an organic EL device including a compound having triphenylene with strong electron tolerance near an amine part with hole transport properties, e.g., in a layer of stacked layers disposed between the emission layer and the anode.

In the organic EL device according to an embodiment, hole transport properties and electron tolerance may be improved, and long life and high efficiency may be realized when using a material for an organic EL device including a compound having triphenylene with strong electron tolerance near an amine part with hole transport properties in the emission layer.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A material for an organic electroluminescence (EL) device, the material including a compound represented by the following Formula (1): 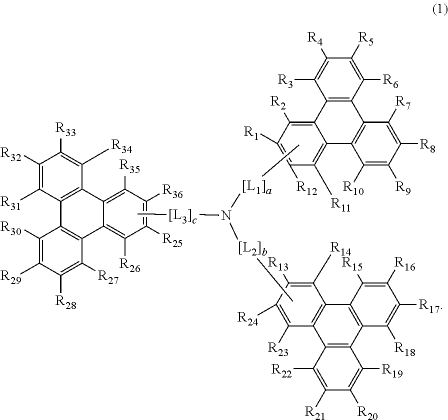
wherein, in Formula (1),
R₁ to R₃₆ are each independently an aryl group having 6 to 30 ring carbon atoms, a heteroaryl group having 1 to 30 ring carbon atoms, an alkyl group having 1 to 15 carbon atoms, a hydrogen atom, a deuterium atom, or a bonding site at which N or a respective one of L₁ to L₃ is bound to a triphenylene ring carbon, and
L₁ to L₃ are each independently a divalent connecting group, where L₁ is combined with one of R₁ to R₁₂, L₂ is combined with one of R₁₃ to R₂₄, and L₃ is combined with one of R₂₅ to R₃₆, and a, b, and c are each independently an integer from 0 to 3, and satisfy the equation 1≧a+b+c.

2. The material as claimed in claim 1, wherein L₁ to L₃ of Formula (1) are each a phenylene group and the compound is represented by the following Formula (2):

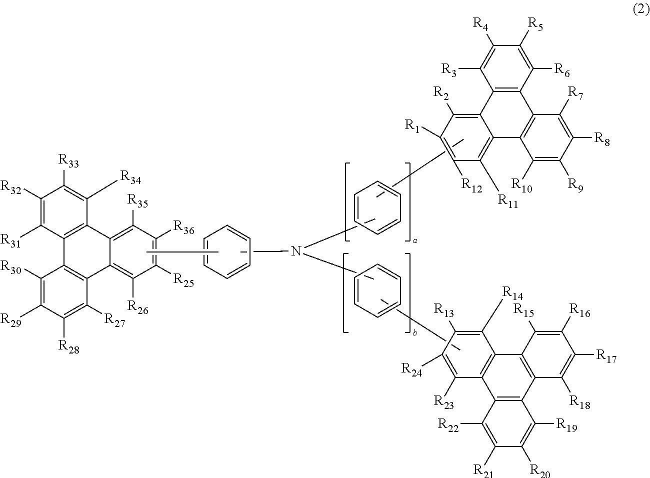

3. The material as claimed in claim 1, wherein a, b, and c satisfy the equation 1≧a+b+c≦2.

4. An organic electroluminescence (EL) device comprising the material as claimed in claim 1 in a layer between an emission layer and an anode.

5. An organic electroluminescence (EL) device comprising the material as claimed in claim 2 in a layer between an emission layer and an anode.

6. An organic electroluminescence (EL) device comprising the material as claimed in claim 1 in an emission layer.

7. An organic electroluminescence (EL) device comprising the material as claimed in claim 2 in an emission layer.
